(19) United States
(12) Patent Application Publication
(10) Pub. No.: US 2021/0228336 A1
(43) Pub. Date: Jul. 29, 2021

(54) Rotational Stable Intraocular Lens Anchored in Asymmetrical Capsulorhexis

(71) Applicant: AKKOLENS INTERNATIONAL B.V., Breda (NL)

(72) Inventors: Michiel Christiaan Rombach, Breda (NL); Aleksey Nikolaevich Simonov, Breda (NL); Willem Pieter Van Lawick, Breda (NL)

(21) Appl. No.: 16/972,185

(22) PCT Filed: May 20, 2019
(86) PCT No.: PCT/NL2019/050291

§ 371 Date: Dec. 4, 2020

(30) Foreign Application Priority Data
Jun. 7, 2018 (NL) 2021079

(51) Int. Cl. A61F 2/16

(52) U.S. Cl. CPC A61F 2/1618; A61F 2002/16901; A61F 2002/169; A61F 2/16015

(57) ABSTRACT

The invention discloses an intraocular lens construction with the lens construction including one or more rotational haptics extending away from the optical component which haptic provide coupling of the lens construction to the rim of at least one asymmetrical capsulorhexis in the capsular bag. The lens construction, fitted with at least one anterior rotational haptic can be positioned inside the capsular bag, or the lens construction fitted with at least one posterior rotational haptic can be positioned at the sulcus plane in front of the capsular bag. Such a lens construction provides rotational stability any type of intraocular lens including any toric intraocular lens.

### CROSS-REFERENCE TO RELATED APPLICATIONS

This application is the United States national phase of International Application No. PCT/NL2019/050291 filed May 20, 2019, and claims priority to The Netherlands Patent Application No. 2021079 filed Jun. 7, 2018, the disclosures of which are hereby incorporated by reference in their entirety.

### BACKGROUND OF THE INVENTION

### Field of the Invention

This invention discloses an intraocular lens construction with the lens construction comprising one or more rotational haptics extending away from the optical component which haptic provide coupling of the lens construction to the rim of at least one asymmetrical capsulorhexis in the capsular bag. The lens construction, fitted with at least one anterior rotational haptic can be positioned inside the capsular bag, or the lens construction fitted with at least one posterior rotational haptic can be positioned at the sulcus plane in front of the capsular bag. Such a lens construction provides rotational stability any type of intraocular lens including any toric intraocular lens.

### Description of Related Art

The natural lens of the human eye can be replaced by an artificial intraocular lens to treat degraded vision due to, for example, a cataract, presbyopia or severe myopia. Intraocular lenses can be implanted in the anterior chamber of the eye, directly behind the cornea, or, alternatively, in the posterior chamber of the eye, behind the cornea and behind the iris, with such posterior implanted lenses positioned in front of the capsular bag, or, alternatively, inside the capsular bag from which the natural lens is surgically removed.

Rotation, with rotation meaning movement in a plane perpendicular to the optical axis, of such lenses in the eye is generally undesirable, especially rotation of lenses with asymmetrical rotational optical properties is undesirable. For example, rotation of a monofocal lens or an annular design multifocal lens has generally no significant effect on its optical performance. However, rotation of a rotational asymmetrical lens, for example, a lens with a cylinder, a ‘toric’ lens, to correct for a cylindrical aberration due to the cornea, or, for example, a bifocal lens with split optics degrades optical performance. Such rotational asymmetrical lenses clearly must remain in a stable rotational position, within a few radial degrees, according to the desired predetermined rotational angle. The present document discloses an intraocular lens construction for a rotational stable intraocular lens.

Anchors, ‘haptics’, of such lenses traditionally provide the desired rotational mechanical stability. Such anchors fixate the lens to the sclera of the eye for lenses positioned in the anterior chamber, or, for lenses in the posterior chamber, to the sclera at the sulcus plane in front of the capsular bag, or, in the posterior chamber, for lenses inside the capsular bag, to the inside rim of the capsular bag.

BR-112016001952 discloses a lens structure to couple the haptics of the lens to the rim of the capsulorhexis, the hole in the anterior section of the bag through which the surgeon removes the natural lens of the eye. As the capsulorhexis is circular, round, and is located in the centre, this prior art likely prevents only decentration of the lens versus the optical axis but not rotation of the lens. Said prior art document discloses coupling of lens optics to a capsulorhexis, to the rim of the rhexis, and it makes no mention of asymmetric capsulorhexis nor of any intention to affect rotational stability of the lens structure. Also, this document BR-112016001952 restricts the invention to only intraocular lenses implanted inside the capsular bag.

U.S. Pat. No. 6,027,531 discloses “An intraocular lens for use in extracapsular cataract extraction has a haptic pa[r]t that surrounds the optical pa[r]t of the lens and further contains a groove of such shape to accommodate the anterior and posterior capsules of the lens bag after anterior capsulorhexis, extracapsular cataract extraction and posterior capsulorhexis. The lens is preferably inserted in a calibrated, circular and continuous combined anterior and posterior capsulorhexis, slightly smaller than the inner circumference of the groove as to induce a stretching of the rims of the capsular openings. This new approach is believed to prevent the appearance of secondary opacification of the capsules, allows a very stable fixation of the intraocular lens and ensures a tight separation between the anterior and posterior segment of the eye. This new principle of insertion is called the bag-in-the-lens technique, in contrast with the classical lens in-the-bag technique.”. Placement of this IOL requires skills and the capsular bag may get damaged. If after insertion the capsular bag ruptures, the IOL will not maintain its position. This document also discloses coupling of lens optics to a circular capsulorhexis and makes no mention of asymmetric capsulorhexis nor of rotational stability of the lens structure.

Also, this U.S. Pat. No. 6,027,531 document restricts the invention to intraocular lenses implanted inside the capsular bag.

In U.S. Pat. No. 6,881,225, an intraocular lens structure for reducing complications is described. According to the abstract, the intraocular lens structure comprises an optic, a support and a closing fixture. The closing fixture is a groove or a valley formed on the side portion of the optic of the intraocular lens. The valley is formed by the optic and a protrusion projecting posteriorly from the optic. The groove or the valley in the optic is made engaged with the posterior capsular opening generally over the entire circumference of the groove or the valley to close the opening of the posterior capsule. Like most of the current IOL structures, the structure also uses its haptics for keeping the structure in the capsular bag. The groove holds the posterior part of the capsular bag. This document also discloses coupling of lens optics to a circular capsulorhexis and makes no mention of asymmetric capsulorhexis nor of rotational stability of the lens structure. Further this document U.S. Pat. No. 6,881,225 is restricted to intraocular lenses implanted into the capsular bag.

U.S. Pat. No. 5,171,320 describes in its abstract an intraocular lens system adapted to be implanted within a generally circular opening in an anterior wall of the capsular bag which normally contains the crystalline lens of an eye. The intraocular lens system includes a lens body having an annular groove which is formed in a peripheral portion thereof in a plane substantially perpendicular to an optical axis of the lens body. The lens body includes an optically effective portion located radially inside the annular groove, and an anterior lens portion and a posterior lens portion located on respective anterior and posterior sides of the annular groove. The intraocular lens system is secured in position within the circular opening such that an annular flap portion of the capsular bag which surrounds the circular opening is accommodated within the annular groove in the lens body. This document discloses coupling of lens optics to a circular, annular, capsulorhexis and makes no mention of asymmetric capsulorhexis nor of rotational stability of the lens structure. Further this document U.S. Pat. No. 5,171,320 restricts the invention to intraocular lenses implanted into the capsular bag.

EP-2 422 746 discloses according to its abstract an intraocular implant for placement in the eye, e.g. as part of a cataract operation or crystalline lens extraction refractive operation, has at a peripheral portion of the implant a groove which engages with the lip of a single capsulotomy only formed in the lens capsule of the eye. The implant will normally be a lens, but may instead be a bung or plug for occluding an opening made in the capsule. The groove may be a continuous groove around the periphery of the implant, or there may be a series of individual spaced-apart grooves formed as projections protruding from the periphery. Instead of a single groove, a pair of axially spaced-apart grooves may be provided, which engage with respective capsulotomies formed in an anterior and a posterior part of the capsule. The posterior groove is preferably of a smaller mean diameter than the anterior groove. The description shows an embodiment with “a series of projections projecting from the circumference of the lens portion”, referring to very specific embodiments in the drawings. This document EP-2 422 746 also discloses the coupling of lens optics to a circular capsulorhexis, but it does not mention asymmetric capsulorhexis nor rotational stability of the lens structure and this document restricts the invention to intraocular lenses implanted into the capsular bag.

### SUMMARY OF THE INVENTION

The present invention relates to a lens construction of a rotational stable intraocular lens, that is a lens construction to be implanted in the eye with the lens construction comprising a combination of at least one rotation haptic component which haptic provides coupling of the lens construction to at least one asymmetrical capsulorhexis and optics, which optics can be any type of optics. So, the rotational stable intraocular lens construction to be implanted in the eye comprises at least one optical component adapted to provide correction of at least one optical aberration of the eye and the lens construction also comprises at least one rotation haptic component which provides anchoring the lens construction in at least one asymmetrical capsulorhexis in the capsular bag. An asymmetrical capsulorhexis is meant to be a capsulorhexis which is located asymetrically relative to the optical axis in the capsular bag.

Note that also two capsulorhexi, one in the anterior segment and one in the posterior segment possibly of a different shape, may be applied. In the present document we focus on one anterior capsulorhexis to explain and illustrate the details of the invention related to only the anterior segment of the capsular bag. As stated before, capsulorhexi may also be applied in the posterior segment of the capsular bag, and the haptics may comprise parts adapted to engage these as well. Additional haptics, for example haptics which provide any other mechanical support can be combined with said rotation haptic in any combination of additional haptics.

The present invention has only recently become technically possible because such a precise asymmetrical capsulorhexis was impossible to provide consistently by traditional manual surgical methods, meaning: manual cutting and pulling of the capsular flap, with ‘flap’ meaning: the anterior section of the bag to be removed by forceps and hooks. However, with the recent introduction of ophthalmological surgical lasers providing surgical assisted capsulotomy almost any shape of capsulorhexis can be provided, and it is likely that we see soon a cut-out in the shape of, say, Cleopatra's nose, and the manual procedure left is just the removal of the capsular cut-out after cutting of the flap by said surgical laser.

A laser assisted capsulotomies provides a sturdy rim of the capsular bag as shown by various scientific studies. Capsulotomies performed by an optical coherence tomography-guided femtosecond laser were evaluated in porcine and human cadaver eyes. Subsequently, the procedure was performed in 39 patients as part of a prospective randomized study of femtosecond laser-assisted cataract surgery. The accuracy of the capsulotomy size, shape, and centration were quantified and capsulotomy strength was assessed in the porcine eyes. Laser-created capsulotomies were significantly more precise in size and shape than manually created capsulorhexes. In the patient eyes, the deviation from the intended diameter of the resected capsule disk was 29 μπι±26 (SD) for the laser technique and 337±258 μπι for the manual technique. The mean deviation from circularity was 6% and 20%, respectively. The centre of the laser capsulotomies was within 77±47 μπι of the intended position. All capsulotomies were complete, with no radial nicks or tears. The strength of laser capsulotomies (porcine subgroup) decreased with increasing pulse energy: 152±21 mN for 3 mJ, 121±16 mN for 6 mJ, and 113±23 mN for 10 mJ. The strength of the manual capsulorhexes was 65±21 mN.

The femtosecond laser produced capsulotomies that were more precise, accurate, reproducible, and stronger than those created with the conventional manual technique. Ten fresh pig eyes were randomly assigned to femtosecond laser-assisted capsulotomy or manual capsulotomy. The capsule was immersed in hyaluronic acid, and retractors were fixed in the capsule opening with a pull-force measuring device. The force necessary to break the capsulotomy was measured in millinewtons (mN); the maximum stretching ratio was also assessed. The observed mean rupture force (i.e., maximum amount of force measured immediately before tissue rupture) was 113 mN±12 (SD) in the laser-assisted procedure and 73±22 mN in the manual procedure (P<05). The stretching ratios were 1.60±0.10 (femtosecond) and 1.35±0.04 (manual) (P<05). (J. Cataract Refract. Surg. 2011; 37: 1189-1198 Q 2011 ASCRS and ESCRS). In another laboratory pig-eye study, femtosecond laser-assisted capsulotomy resulted in a significantly stronger anterior capsule opening than the standard manually performed capsulotomy (J. Cataract Refract. Surg. 2013; 39: 105-109 Q 2013 ASCRS and ESCRS).

In yet another study capsulotomies performed by an optical coherence tomography-guided femtosecond laser were evaluated in porcine and human cadaver eyes. Subsequently, the procedure was performed in 39 patients as part of a prospective randomized study of femtosecond laser-assisted cataract surgery. The accuracy of the capsulotomy size, shape, and centration were quantified and capsulotomy strength was assessed in the porcine eyes. Laser-created capsulotomies were significantly more precise in size and shape than manually created capsulorhexes. In the patient eyes, the deviation from the intended diameter of the resected capsule disk was 29 μπι±26 (SD) for the laser technique and 337±258 μπι for the manual technique. The mean deviation from circularity was 6% and 20%, respectively. The centre of the laser capsulotomies was within 77±47 μπι of the intended position. All capsulotomies were complete, with no radial nicks or tears. The strength of laser capsulotomies (porcine subgroup) decreased with increasing pulse energy: 152±21 mN for 3 mJ, 121±16 mN for 6 mJ, and 113±23 mN for 10 mJ. The strength of the manual capsulorhexes was 65±21 mN. The femtosecond laser produced capsulotomies that were more precise, accurate, reproducible, and stronger than those created with the conventional manual technique. (J. Cataract Refract. Surg. 2011; 37: 1189-1198 Q 2011 ASCRS and ESCRS).

In yet another study ten fresh pig eyes were randomly assigned to femtosecond laser-assisted capsulotomy or manual capsulotomy. The capsule was immersed in hyaluronic acid, and retractors were fixed in the capsule opening with a pull-force measuring device. The force necessary to break the capsulotomy was measured in millinewtons (mN); the maximum stretching ratio was also assessed. The observed mean rupture force (i.e., maximum amount of force measured immediately before tissue rupture) was 113 mN±12 (SD) in the laser-assisted procedure and 73±22 mN in the manual procedure (P<05). The stretching ratios were 1.60±0.10 (femtosecond) and 1.35±0.04 (manual) (P<05). In this laboratory pig-eye study, femtosecond laser-assisted capsulotomy resulted in a significantly stronger anterior capsule opening than the standard manually performed capsulotomy. (J. Cataract Refract. Surg. 2013; 39: 105-109 Q 2013 ASCRS and ESCRS).

The invention disclosed in the present document provides a rotational stable intraocular lens construction, adapted to be implanted in an eye, the lens construction comprising at least one optical component, adapted to provide correction of at least one optical aberration of the eye and haptics adapted to position the lens construction in the eye, in which the haptics comprise at least one rotation haptic, adapted to provide coupling of the lens construction to at an capsulorhexis provided in an asymmetrical position in the capsular bag.

In a first embodiment the lens construction is adapted to be implanted in the capsular bag and that the lens construction comprises at least one anterior haptic which haptic is adapted to provide coupling of the lens construction to the capsulorhexis which is provided in the anterior section of the capsular bag.

Preferably the lens construction is adapted to be implanted at the sulcus plane, the lens construction comprising at least one posterior haptic adapted to provide anchoring of the lens construction to the capsulorhexis which is provided in the anterior section of the capsular bag. The expression ‘sulcus plane’ is used to indicate the plane behind the iris of the eye and in front of the capsular bag.

In another embodiment the lens construction comprises at least one posterior haptic, which haptic is adapted to provide coupling of the construction to a capsulorhexis which is provided in the posterior section of the capsular bag.

According to a specific embodiment the parts of the lens construction are composed of the same material. These parts by be separate parts, or may be united to a single part. During surgery the element with haptics is, for example, positioned first to be coupled to the element with the optics thereafter.

It is however also possible that the parts of the lens construction are composed of different materials, such as one material forming the haptics and the other element the optics, for example PMMA for the element comprising the haptics and an acrylic material comprising the optics. Alternatively, of different materials, with one element comprising the haptics and the other the optics. During surgery the element with haptics is, for example, positioned first to be coupled to the element with the optics thereafter. So, the lens construction can be composed of a combination of at least two components with at least one component comprising the optics and at least one other component comprising the haptics with said components to be coupled in the eye before or, alternatively, during the surgery to ease, for example, surgical positioning of the posterior haptics.

The angle of the rotational asymmetrical anterior capsulorhexis provides anchoring of the lens construction at a desired mechanically rotational angle, an angle based on, for example, the asymmetrical shape of the sulcus of the eye, or, alternatively, or, alternatively, the angle of the rotational asymmetrical anterior capsulorhexis provides anchoring of the lens construction at a desired optically rotational angle, an angle based on, for example, the desired angle of a toric optics to correct for a cylinder of the cornea.

Preferably the lens construction comprises at least one monofocal optical component comprising at least one optical surface which component is adapted to provide correction of defocus of the eye.

It is however also possible that the lens construction comprises at least one multifocal optical component comprising at least one optical surface which component is adapted to provide correction of defocus of the eye.

As an alternative the lens construction comprises at least one extended depth of field optical component comprising at least one optical surface which component is adapted to provide correction of defocus of the eye.

Yet it is also possible that the lens construction comprises also at least one accommodative optical component comprising at least one optical surface which component is adapted to provide correction of defocus of the eye, that the lens construction comprises at least one additional optical surface which surface is adapted to correct for at least one additional optical aberration other than defocus or that the lens construction comprises at least one additional optical surface adapted to provide correction of astigmatism of the eye.

### BRIEF DESCRIPTION OF THE DRAWINGS

Subsequently the present invention will be elucidated with the help of the following drawings, showing:

FIG. 1: A front view of the remaining rim of the capsular bag, 1, provided with a, traditional round, symmetrical, capsulorhexis, 2, and rotation haptics, 3. In such embodiment the capsulorhexis shape allows rotation of the lens, see the arrows, 4;

FIG. 2: A front view: Capsular bag rim, 1, provided with an oval asymmetrical capsulorhexis, 5, and rotation haptics, 6. In such embodiment the shape of the capsulorhexis prevents rotation, see the arrows, 7;

FIG. 3: A front view: As FIG. 2, with the capsular bag provided with an aligned double-oval shaped capsulorhexis, 8, which shape prevents rotation, see arrows. Note: FIG. 1-3 provide a view perpendicular to the optical axis, that is a frontal view, which view is similar for a lens construction with anterior rotation haptics and posterior rotation haptics;

FIG. 4: A side view: The lens construction, in this example comprising a single optic, for example, a monofocal toric lens, implanted in the eye inside the capsular bag with the eye with cornea, 9, the anterior chamber of the eye, 10, the iris, 11, a lens construction, 12, the capsular bag and capsulorhexis, 13. The two posterior haptics, 16, anchor the lens construction in the, asymmetric, capsulorhexis;

FIG. 5: A side view, for further anatomical details see FIG. 4: The lens construction implanted in the eye at the sulcus plane The two anterior haptics, 14, anchor the lens construction in the, asymmetric, capsulorhexis;

FIG. 6: A side view, for further anatomical details see FIG. 4: As FIG. 4, but with a construction positioned though a capsulorhexis in both the anterior and the posterior segments of the capsule with the construction with anterior rotation haptics;

FIG. 7: A side view, for further anatomical details see FIG. 4: An example of a lens construction with both anterior and posterior rotation haptics; and

FIG. 8: A side view, for further anatomical details see FIG. 4: Lens construction, in this example comprising a double element optic, 15, for example, an accommodating lens according to for example NL2012133 and US2012310341 which is implanted in the eye at the sulcus plane and with accommodation driven by the ciliary mass and ciliary muscle, 16, in a direction perpendicular to the optical axis, see arrows, 17. The two posterior haptics, 18, anchor this lens construction in the, asymmetric, capsulorhexis.

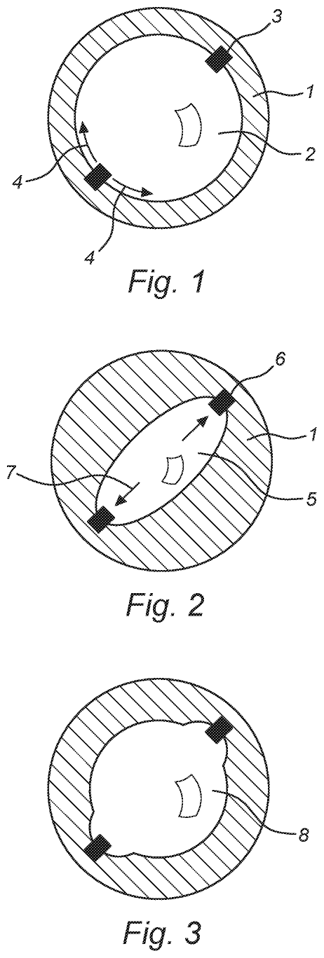

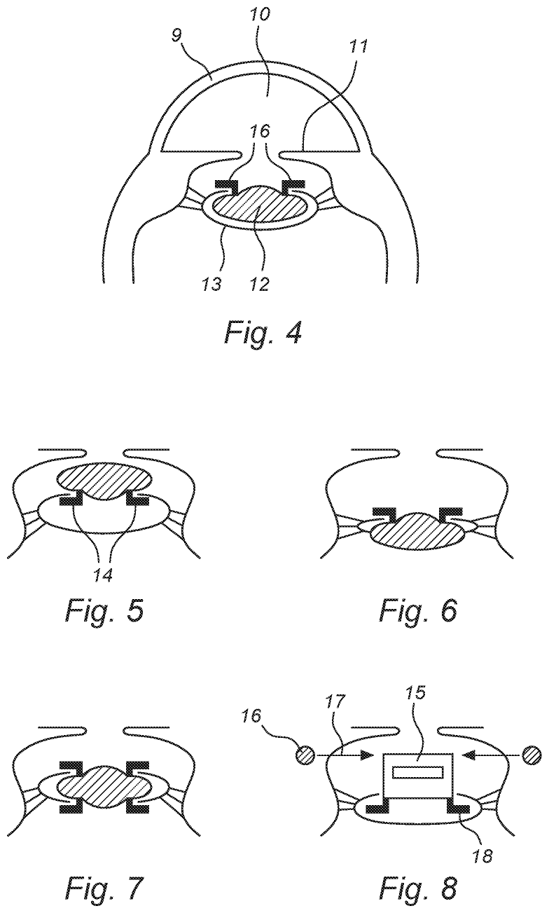

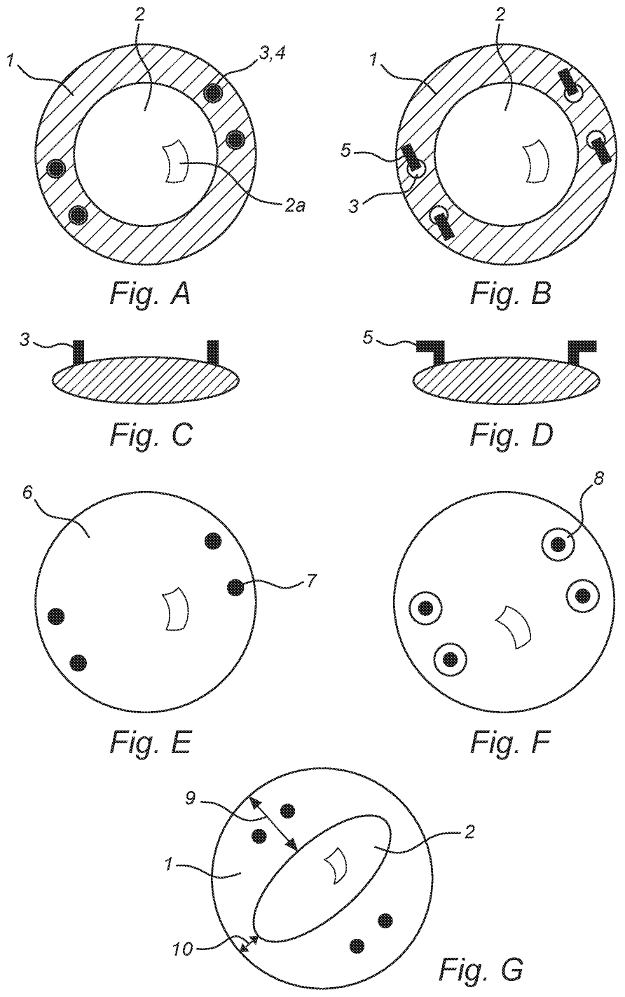

### DESCRIPTION OF THE INVENTION

The rotation haptics can comprise hook-shaped components, or, alternatively, clamp-shaped components, or, alternatively, ridge-shaped components, or, alternatively, slit-shaped components, tapered-shaped component, or, alternatively, any other shaped components to couple the rotation haptic to the rim of the capsulorhexis. All said haptic shapes can be mutually shifted or staggered.

The lens construction can comprise monofocal optics which can also can comprise a toric optical component. Alternatively, the lens construction can comprise multifocal optics which can also can comprise a toric optical component. Alternatively, the lens construction can comprise extended depth of field, DOF, optics which can also can comprise a toric optical component. Alternatively, the lens construction can comprise accommodative which can also comprise a toric optical component. The lens can be construction is composed of a combination of at least two components with at least one component, the optical component, comprising the optics and at least one other component, the mechanical component, comprising the haptics.

So, regarding optics, the lens construction can comprise various optical embodiments, such as at least one monofocal optical component comprising at least one optical surface which component is adapted to provide correction of defocus of the eye, or, alternatively, the construction can comprise at least one multifocal optical component comprising at least one optical surface which component is adapted to provide correction of defocus of the eye, or, alternatively, the construction can comprise at least one extended depth of field optical component comprising at least one optical surface which component is adapted to provide correction of defocus of the eye. The construction can comprises also at least one accommodative optical component comprising at least one optical surface which component is adapted to provide correction of defocus of the eye, with all of these optical embodiments can also comprise at least one additional optical surface which surface is adapted to correct for at least one additional optical aberration other than defocus, for example, with said surface at least one additional optical surface adapted to provide correction of astigmatism of the eye.

The method related to said rotationally stable intraocular lenses comprises providing at least one asymmetrical capsulorhexis, and fixation of the construction into an eye with the method in turn comprising forming an asymmetric opening, capsulorhexis, in an anterior and/or posterior part of a capsular bag of an eye, in particular performing a laser-assisted capsulotomy, and, removing a natural lens from the capsular bag through said opening, and, inserting the construction inside the bag through said opening or, alternatively, in front of the capsular bag, and, taking the anterior haptic components out the capsular bag while leaving the optical component inside the capsular bag, or, alternatively, positioning the posterior haptic components inside the capsular bag while leaving the optical component outside the capsular bag.

The description and drawings illustrate some embodiments of the invention, and do not limit the scope of protection. Many more embodiments will be evident to a skilled person. These embodiments are within the scope of protection and the essence of this invention and are obvious combinations of techniques and the disclosure of this patent.

So, in summary, this invention discloses an intraocular lens construction being adapted to be implanted in the with the lens construction comprising at least one rotation haptic extending with a component in the direction of the optical axis and which haptic is adapted to provide coupling the lens to, anchoring the lens, to at least one natural structure of the eye, for example, to at least one asymetrically shaped capsulorhexis in the capsular bag of the eye, with the lens construction adapted to be implanted in the capsular bag and that the at least one rotation haptic extending in the anterior direction and is adapted to provide anchoring the lens construction in the asymmetrical anterior capsulorhexis, or, alternatively, with the lens construction adapted to be implanted anterior, in front of, of the capsular bag, with the rotation haptic extending in the posterior direction and that the haptic is adapted to provide anchoring the lens construction in the asymmetrical anterior capsulorhexis, with the angle of the rotational asymmetrical anterior capsulorhexis adapted to provide

anchoring of the lens construction at a mechanically rotational angle, or, alternatively, with the angle of the rotational asymmetrical anterior capsulorhexis adapted to provide

anchoring of the lens construction at an optically rotational angle, with the lens construction comprises monofocal optics, or, alternatively, with the lens

construction comprising multifocal optics, or, alternatively, with the lens

construction comprising extended depth of field, DOF, optics, or, alternatively, with construction comprising accommodative optics.

In addition to the above invention, and fully independent from the above invention, it is also disclosed that a rotationally stable intraocular lens can be provided by a rotation ‘pin-in-hole’ configuration, or, alternatively, ‘clamp-in-hole’ configuration, or, alternatively, ‘rivet-in-hole’ configuration, or, alternatively, any other shaped coupling configuration, henceforth configurations referred to as ‘pin-in-hole’, coupling the construction to the capsular bag not at the rim of the bag as outlined above, but in the rim of the bag, meaning: in the remaining ring in between the zonulae and the capsulorhexis. Such ‘pin-in-hole’ rotational stable intraocular lens construction’, also: ‘construction’, to be implanted in the eye with the lens construction comprising at least one optical component, also: ‘optics’, adapted to provide correction of at least one optical aberration of the eye with the lens also comprises at least one rotation pin which pin is adapted to fit, couple to, in at least hole in the rim of the anterior capsular bag, or, alternatively, in the posterior capsular bag, which can have a rim, or, alternatively, in holes in both the anterior and the posterior bag. Such construction does not necessarily requires a asymmetrical capsulorhexis which capsulorhexis can also be circular, annular. However, a asymmetrical capsulorhexis, for example, an oval rhexis, can provide additional space for pins or rivets at the smallest diameter of the rhexis. Holes and pins can be provided to the rim of the anterior capsule, or, alternatively, to the rom of the posterior capsule, or alternatively, to the rims of both capsules. Clearly, laser assisted capsulotomy is the preferred method to provide for capsulorhexis and, specifically, for said holes in the capsular tissue which can not be provided by manual surgery.

The method related to said ‘pin-in-hole’ rotationally stable intraocular lenses comprises providing at least one asymmetrical capsulorhexis and at least one, preferably two or more, small holes of any shape in a rim of the capsular bags by laser assisted capsulotomy, a shape favourable for fitting the pins, removing a natural lens from the capsular bag through said opening, and, inserting the construction inside the bag through said opening or, alternatively, in front of the capsular bag, rotation and manipulation to fit pins in the holes followed by traditional surgicals irrigation and cleaning.

Figure A. Front view: Capsular bag remaining rim, 1, and capsulorhexis, 2, and reflection on the optical component of the lens, 2a, and in this example the pins connected to the lens construction fitted in the holes in the capsular rim.

Figure B. As Figure A, but with clamps which couple the construction to the rim of the capsular bag.

Figure C. Side view: Lens construction comprising pins.

Figure D. Side view: Lens construction comprising clamps.

Figure E. Front view: Lens construction with pins.

Figure F. Front view of lens construction with rivets.

Figure G. Construction and coupling as outlined in Figures A-F, but with a asymmetrical capsulorhexis which allow pins or rivets to be poisoned in the preferable widest section of the remaining rim of the bag, see arrows 9 and 10.

So, the present document discloses a rotational stable intraocular lens construction, to be implanted in an eye, with the lens construction comprising at least one optical component to provide correction of at least one optical aberration of the eye and haptics to position the lens construction in the eye with the haptics comprising at least one rotation haptic to provide coupling of the lens construction to an asymmetrical capsulorhexis provided in the capsular bag. The lens construction can be adapted to be implanted in the capsular bag with the lens construction comprising at least one anterior haptic which haptic provides coupling of the lens construction to the capsulorhexis which is provided in the anterior section of the capsular bag, or, alternatively, the lens construction can be adapted to be implanted at the sulcus plane, the plane behind the iris of the eye and in front of the capsular bag, with the lens construction comprising at least one posterior haptic to provide anchoring of the lens construction to the capsulorhexis which is provided in the anterior section of the capsular bag, or, alternatively, the lens construction can comprises at least one posterior haptic to provide coupling of the construction to a capsulorhexis which is provided in the posterior section of the capsular bag. The lens construction comprises an optical component, comprising the optics and a mechanical component, comprising the haptics and the construction can be composed of the same material, or, alternatively, the lens construction can be composed of different materials.

The lens construction can comprise at least one monofocal optical component comprising at least one optical surface which component provides correction of defocus of the eye, or, alternatively, the lens construction can comprise at least one multifocal optical component comprising at least one optical surface which component provides correction of defocus of the eye, or, alternatively, the lens construction comprises at least one extended depth of field optical component comprising at least one optical surface which component to provides correction of defocus of the eye, or, alternatively, the lens construction comprises also at least one accommodative optical component comprising at least one optical surface which component provides correction of defocus of the eye with such lens constructions can also comprises at least one additional optical surface which surface provide to correction of at least one additional optical aberration f the eye other than defocus which the additional optical surface providing, for example, correction of astigmatism of the eye.

The method for providing a lens construction according to one of the preceding claims in the eye can comprise providing a asymmetrical capsulorhexis, in an anterior and/or posterior part of a capsular bag of an eye, in particular performing a laser-assisted capsulotomy, and that the method further comprises the following steps:

removing a natural lens from the capsular bag through said capsulorhexis, inserting the lens construction into the capsular bag through said capsulorhexis or, alternatively, inserting the lens construction anterior of the capsular bag, taking at least a section of the rotation haptic out the capsular bag while leaving the optical component inside the capsular bag, or, alternatively, positioning the rotation haptic inside the capsular bag while leaving the optical component outside the capsular bag.

1. A rotational stable intraocular lens construction, adapted to be implanted in an eye, the lens construction comprising at least one optical component, adapted to provide correction of at least one optical aberration of the eye and haptics adapted to position the lens construction in the eye, characterized in that the haptics comprise at least one rotation haptic, adapted to provide coupling of the lens construction to an asymmetrical shaped capsulorhexis provided in the capsular bag.

2. The lens construction according to claim 1, characterized in that the lens construction is adapted to be implanted in the capsular bag and that the lens construction comprises at least one anterior haptic which haptic is adapted to provide coupling of the lens construction to the capsulorhexis which is provided in the anterior section of the capsular bag.

3. The lens construction according to claim 1, characterized in that the lens construction is adapted to be implanted at the sulcus plane, the plane behind the iris of the eye and in front of the capsular bag, with the lens construction comprising at least one posterior haptic adapted to provide anchoring of the lens construction to the capsulorhexis which is provided in the anterior section of the capsular bag.

4. The lens construction according to claim 2, characterized in that the lens construction comprises at least one posterior haptic, which haptic is adapted to provide coupling of the construction to a capsulorhexis which is provided in the posterior section of the capsular bag.

5. The lens construction according to claim 1, characterized in that the parts of the lens construction are composed of the same material.

6. The lens construction according to claim 1, characterized in that the parts of the lens construction are composed of different materials.

7. The lens construction according to claim 1, characterized in that construction is comprises an optical component, comprising the optics and a mechanical component, comprising the haptics.

8. The lens construction according to claim 1, characterized in that the lens construction comprises at least one monofocal optical component comprising at least one optical surface which component is adapted to provide correction of defocus of the eye.

9. The lens construction according to claim 1, characterized in that the lens construction comprises at least one multifocal optical component comprising at least one optical surface which component is adapted to provide correction of defocus of the eye.

10. The lens construction according to claim 1, characterized in that the lens construction comprises at least one extended depth of field optical component comprising at least one optical surface which component is adapted to provide correction of defocus of the eye.

11. The lens construction according to claim 1, characterized in that the lens construction comprises also at least one accommodative optical component comprising at least one optical surface which component is adapted to provide correction of defocus of the eye.

12. The lens construction according to claim 8, characterized in that the lens construction comprises at least one additional optical surface which surface is adapted to correct for at least one additional optical aberration other than defocus.

13. The lens construction according to claim 12, characterized in that the lens construction comprises at least one additional optical surface adapted to provide correction of astigmatism of the eye.

14. The method for providing a lens construction according to claim 1 in the eye, characterized in that that the method comprises providing a asymmetrical capsulorhexis.

15. The method according to claim 14, characterized in that that the capsulorhexis is provided in an anterior and/or posterior part of a capsular bag of an eye, in particular performing a laser-assisted capsulotomy, and that the method further comprises the following steps:
removing a natural lens from the capsular bag through said capsulorhexis,
inserting the lens construction into the capsular bag through said capsulorhexis or, alternatively, anterior to the capsular bag,
taking at least a section of the rotation haptic out the capsular bag while leaving the optical component inside the capsular bag, or, alternatively,
positioning the rotation haptic inside the capsular bag while leaving the optical component outside the capsular bag.
